(19) United States
(12) Patent Application Publication
(10) Pub. No.: US 2015/0360038 A1
(43) Pub. Date: Dec. 17, 2015

(54) Heads-Up Display and Control of an Implantable Medical Device

(71) Applicant: Boston Scientific Neuromodulation Corporation, Valencia, CA (US)

(72) Inventors: Dennis Zottola, Ventura, CA (US); Stephen Carcieri, Los Angeles, CA (US)

(21) Appl. No.: 14/710,283

(22) Filed: May 12, 2015

Related U.S. Application Data
(60) Provisional application No. 62/103,331, filed on Jan. 14, 2015.
(60) Provisional application No. 62/033,204, filed on Aug. 5, 2014.
(60) Provisional application No. 62/011,577, filed on Jun. 13, 2014.

(51) Int. Cl. A61N 1/372

(52) U.S. Cl. CPC A61N 1/37247; A61N 1/37217; A61N 1/37264

(57) ABSTRACT

A clinician programmer (CP) system for programming a patient's Implantable Medical Device (IMD) is disclosed having an optical head-mounted display (OHMD) that a clinician can use to adjust the therapy provided by the IMD, such as the stimulation parameters provided by an Implantable Pulse Generator (IPG). The OHMD is preferably enabled by improved CP software operable in a CP system computer to render an OHMD Graphical User Interface (GUI) in the OHMD, which may be limited to critical CP functionality; non-critical functionality can be rendered by the CP software on the CP computer. The OHMD GUI is preferably rendered in a simple format within the clinician's field of view. The clinician can access the OHMD GUI, by touch or voice for example, to change therapy parameters and to send such changes to the patient's IMD while continuing to observe the patient.

### CROSS-REFERENCE TO RELATED APPLICATIONS

This non-provisional patent application claims priority to U.S. Provisional Patent Application Ser. Nos. 62/103,331, filed Jan. 14, 2015; 62/033,204, filed Aug. 5, 2014; and 62/011,577, filed Jun. 13, 2014. Priority is claimed to these applications and they are incorporated herein by reference in their entireties.

### FIELD OF THE INVENTION

This application relates to Implantable Medical Devices (IMDs) generally, deep brain stimulators more specifically, and to methods of control of such devices using a heads-up display.

### BACKGROUND

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc.

As shown in FIG. 1, an Implantable Pulse Generator (IPG) 10 includes a biocompatible case 12 formed of a conductive material such as titanium for example. As shown in the cross sections of the IPG 10 in FIGS. 2A and 2B, the case 12 typically holds a printed circuit board (PCB) 14 to which circuitry 16 is coupled (e.g., various integrated circuits, control circuitry (e.g., a microcontroller), capacitors, temperature sensors, etc.) as required by the functionality of the IPG. The IPG's case 12 as depicted also contains a battery 18 to power the circuitry 16, although IPGs can also be continuously powered via an external wireless energy source (not shown).

As shown in FIG. 1, the IPG 10 is usually coupled to a plurality of leads (two of which, 20 and 22, are shown) each containing several electrodes 24, which in sum comprise an electrode array 26 with electrodes E1-E16. The electrodes 24 are carried on flexible lead bodies 28, which also house the individual lead wires 30 coupled to each electrode 24. There are eight electrodes on each lead 20 and 22 in the depicted example, although the number of leads and electrodes is application specific and therefore can vary. The conductive case 12 can also comprise an electrode (Ecase) as explained later. The leads 20 and 22 couple at their proximal ends to the IPG 10 at lead connectors 32, which are fixed in a non-conductive header material 34 such as an epoxy that is affixed to the case 12. Feedthrough wires 36 (FIGS. 2A and 2B) allow coupling of electrode signals from the circuitry 16 inside the case 12 to the contacts in the lead connectors 32, as is well known.

The IPGs 10 in FIGS. 2A and 2B include a charging coil 38 for receiving a magnetic charging field (e.g., 80 kHz) from an external charger (not shown). A current or voltage is induced by the magnetic charging field in the charging coil 38, which is rectified in the IPG 10 and used to charge the IPG's battery 18.

The IPGs 10 in FIGS. 2A and 2B also include antennas 40a and 40b for transmitting and/or receiving data to and/or from patient external controllers 44a and 44b used to control or monitor IPG operation. The IPGs in FIGS. 2A and 2B though differ in antenna type to accommodate the means of communication supported by the patient external controllers 44a and 44b.

The IPG 10 of FIG. 2A has a coil antenna 40a to enable bi-directional communications with a cooperative coil antenna 46a in external controller 44a via near-field magnetic induction. The transmitting coil antenna (40a or 46a) generates a magnetic field 42a modulated with data. Such modulation can occur for example using Frequency Shift Keying (FSK), in which ‘0’ and ‘1’ data bits comprise frequency-shifted values (e.g., f0=121 kHz, f1=129 kHz) with respect to the center frequency of the magnetic field 42a (e.g., fc=125 kHz). The modulated magnetic field 42a induces a current in the receiving coil antenna (46a or 40a), and is demodulated in the receiving device to recover the data. The magnetic field 42a can comprise a frequency of 10 MHz or less and can communicate over distances of 12 inches or less for example.

The coil antenna 40a is depicted inside the case 12 in FIG. 2A, but it may also be mounted in the IPG's header 34. Further, a single coil could be used in the IPG 10 for both charging and data telemetry functions, as disclosed in U.S. Patent Publication 2010/0069992.

The IPG 10 of FIG. 2B has a short-range RF antenna 40b to enable bi-directional communications with a cooperative short-range RF antenna 46b in external controller 44b via far-field electromagnetic waves 42b. Such communications can occur using well-known short-range RF standards, such as Bluetooth, BLE, NFC, Zigbee, WiFi, and the Medical Implant Communication Service (MICS). The IPG short-range RF antenna 40b and modulation/demodulation circuitry to which it is coupled would in this case be compliant with one or more of these standards. Short-range RF antenna 406 could comprise any number of well-known forms for an electromagnetic antenna, such as patches, slots, wires, etc., and can operate as a dipole or a monopole, and with a ground plane as necessary (not shown). The short-range RF link 42a can comprise a frequency ranging from 10 MHz to 10 GHz or so and can communicate over distances of 50 feet or less for example.

Notice that regardless whether the IPG 10 includes a coil antenna 40a (FIG. 2A) or a short-range RF antenna 40b (FIG. 2B), bi-directional wireless communications via links 42a and 42b can occur transcutaneously through the tissue 48 of the patient.

Examples of hand-holdable and portable patient external controllers 44a and 44b to wirelessly control and communicate with IPGs, including the use of commercially-available mobile devices (e.g., cell phones) and intermediary communication bridge devices, are disclosed in U.S. patent application Ser. No. 14/599,743, filed Jan. 19, 2015, which is incorporated herein by reference in its entirety. Although not depicted, note that such external controllers 44a or 44b typically include a graphical user interface (GUI) including a display and touchable buttons (or a touch-sensitive display) similar to that used for mobile devices generally (e.g., smart phones).

Examples of hand-holdable and portable external chargers for charging the IPG's battery 18 via charging coil 38, including removable external charging coils coupleable to other external devices such as patient external controllers, and unified external devices operable as both a controller and charger, are disclosed in U.S. Pat. Nos. 8,682,444, 8,463,392, 8,335,569, and 8,498,716.

The IPGs 10 illustrated in FIGS. 1, 2A and 2B can be used or modified for the treatment of many different types of conditions for which neurostimulation is useful. For example, the IPG can be used as a Spinal Cord Stimulator (SCS). In such an application, the IPG 10 is typically implanted in the upper buttocks of the patient. The leads 20 and 22 (as assisted by lead extensions if necessary) are tunneled under the skin of patient and into the patient's spinal column such that the electrodes 24 on the distal ends of leads 20 and 22 are implanted on the right and left side of the dura for example.

The illustrated IPGs 10 may also be useful in Deep Brain Stimulation (DBS) for the treatment of Parkinson's disease, essential tremor (ET), and other neurological movement disorders. In such an application, the IPG 10 is typically implanted in the chest of a patient, as shown in FIG. 3, or near the base of the skull or any other convenient location. The leads 20 and 22 (as assisted by lead extensions if necessary) are tunneled under the skin of patient. Holes are drilled in the patient's skull, and the distal ends of the leads 20 and 22 are positioned through the holes to bring the electrodes 24 into contact with the patient's brain. Typically, the electrodes 24 of leads 20 and 22 are respectively positioned in right and left sides of the brain, and in particular areas of interest, such as the subthalamic nucleus (STN) and the pedunculopontine nucleus (PPN). Although not shown, four IPG leads can be provided, with two placed in the STN and PPN in the right brain, and two placed in the STN and PPN in the left brain, although this is not shown for simplicity. See, e.g., U.S. Patent Application Publication 2013/0184794 for further details.

Once an IPG 10 has been implanted in a patient, in a DBS application or otherwise, the clinician can adjust various stimulation parameters to arrive at one or more stimulation programs that provide an IPG patient with optimal therapeutic benefit. For example, the clinician can determine which electrodes 24 on the leads 20 and 22 should be active to provide stimulation pulses (Ex), and the polarity of such electrodes (Px), i.e., whether they are to act as anodes to source current to the patients tissue 48, or cathodes to sink current from the tissue. The clinician can also adjust the amplitude (A; current or voltage), duration (D; pulse width), and frequency (F) of the stimulation pulses at those electrodes. Sometimes, arriving at an optimal stimulation program requires assistance from the patient, such as receiving patient feedback whether particular stimulation parameters are providing therapeutic relief or are causing undesired consequences.

The clinician typically uses a clinician programmer (CP) system 50, such as illustrated in FIG. 4, to determine optimal stimulation program(s) for the patient and to monitor IPG operation. As shown, CP system 50 can comprise a computing device 51, such as a desktop, laptop, or notebook computer, a tablet, a mobile smart phone, a Personal Data Assistant (PDA)-type mobile computing device, etc. (hereinafter “CP computer”). In FIG. 4, CP computer 51 is shown as a laptop computer that includes typical computer user interface means such as a screen 52, a mouse, a keyboard, speakers, a stylus, a printer, etc., not all of which are shown for convenience.

Also shown in FIG. 4 are accessory devices for the CP system 50 that are usually specific to its operation as an IPG controller, such as a communication wand 54, and a joystick 58, which are coupleable to suitable ports on the CP computer 51, such as USB ports 59 for example.

The antenna used in the CP system 50 to communicate with the IPG 10 can depend on the data telemetry antenna included in the IPG 10. If the patient's IPG 10 includes a coil antenna 40a (FIG. 2A), the wand 54 can likewise include a coil antenna 56a to establish communication over a magnetic induction link 42a at small distances (much like the coil antenna 46a in the patient external controller 44a of FIG. 2A). In this instance, the wand 54 may be affixed in close proximity to the patient, such as by placing the wand 54 in a belt or holster wearable by the patient and proximate to the patient's IPG 10.

If the IPG 10 includes a short-range RF antenna 40b (FIG. 2B) with a generally longer communication distance, the wand 54, the CP computer 51, or both, can likewise include a short-range RF antenna 56b (much like the short-range RF antenna 46b in the patient external controller 44b of FIG. 2B) to establish communication with the IPG 10 over a short-range RF link 42b at larger distances. (Thus, a CP wand 54 may not be necessary if the IPG 10 has a short-range RF antenna 40b). If the CP system 50 includes a short-range RF antenna 56b, such antenna can also be used to establish communication between the CP system 50 and other devices, and ultimately to larger communication networks such as the Internet, as subsequently discussed. The CP system 50 can typically also communicate with such other networks via a wired link 62 provided at a Ethernet or network port 60 on the CP computer 51, or with other devices or networks using other wired connections (e.g., at USB ports 59).

Joystick 58 is generally used as an input device to select various stimulation parameters (and thus may be redundant of other input devices to the CP), but is also particularly useful in steering currents between electrodes to arrive at an optimal stimulation program, as discussed further below.

To program stimulation parameters, the clinician interfaces with a clinician programmer graphical user interface (CP GUI) 64 provided on the display 52 of the CP computer 51. As one skilled in the art understands, the CP GUI 64 can be rendered by execution of CP software 66 on the CP computer 51, which software may be stored in the CP computer's non-volatile memory 68. One skilled in the art will additionally recognize that execution of the CP software 66 in the CP computer 51 can be facilitated by control circuitry 70 such as a microprocessor, microcomputer, an FPGA, other digital logic structures, etc., which is capable of executing programs in a computing device. Such control circuitry 70 when executing the CP software 66 will in addition to rendering the CP GUI 64 enable communications with the IPG 10 through a suitable IPG-compliant antenna 56a or 56b, either in the wand 54 or the CP computer 51 as explained earlier, so that the clinician can use the CP GUI 64 to communicate the stimulation parameters to the patient's IPG 10.

A portion of the CP GUI 64 is shown in one example in FIG. 5, and shows aspects taken from “Boston Scientific Precision Spectra™ System Programming Manual,” located on line at http://hcp.controlyourpain.com/hcp/assets/File/us/90668528-07RevB_US.pdf, which is incorporated herein by reference. One skilled in the art will understand that the particulars of the CP GUI 64 will depend on where CP software is in its execution, which will depend on the GUI selections the clinician has made.

FIG. 5 shows the CPU GUI 64 as rendered upon execution of the “mapping and programming” module 214 of the CP software 66 which is further explained later with respect to FIG. 9. This module 214 allows for the setting of stimulation parameters for the patient and for their storage as a stimulation program. To the left a program interface 72 is shown, which shows the stimulation program number 74 currently being displayed in the CP GUI 64, a program options menu 76 (allowing for naming, loading and saving of storage programs for the patient), and an on/off button 78 which when on wirelessly provides the (modified) parameters of the program to the IPG 10, or when off suspends stimulation at the IPG. A program area interface 80 provides four program areas (A-D) to allow stimulation parameters in the program to be defined for particular areas of the body. For example, area A may comprise aspects of the stimulation program for treating lower back pain, while area B may comprise aspects for treating leg pain. Basic waveform stimulation parameters for each of the areas 78 (A, D, F) can be displayed, and each area's stimulation can selectively be turned on or off with a button as shown.

Shown to the right is a stimulation parameters interface 82, in which specific stimulation parameters (A, D, F, Ex, Px) can be defined for the stimulation program (or for an area 80 of the program). Values for stimulation parameters relating to the shape of the waveform (A; in this example, current), duration or pulse width (D), and frequency (F) are shown in a waveform parameter interface 84 of the parameters interface 82, including buttons the clinician can use to increase or decrease these values.

Stimulation parameters relating to the electrodes 24 (the electrodes Ex chosen to receive the waveform parameters specified in module 84, their polarities Px, and relative strengths), are made adjustable in an electrode parameter interface 86 of the parameters interface 80, and these electrode stimulation parameters are also visible and can be manipulated in a leads interface 92. For example, a cursor 94 (or other selection means such as a mouse pointer) can be used to select a particular electrode 24 in the leads interface 92. Buttons in the electrode parameter interface 86 allow the selected electrode (including the case electrode, Ecase) to be designated as an anode, a cathode, or off (It is assumed here that leads 20 and 22 have been previously associated into a “lead group,” which is discussed later).

The electrode parameter interface 86 further allows the relative strength of anodic or cathodic current of the selected electrode to be specified in terms of a percentage. This is particularly useful if more than one electrode is to act as an anode or cathode at a given time. For example, as shown in the leads interface 92, electrode E4 has been selected as the only cathode to sink current (the minus sign in 96), but both of electrodes E2 and E5 have been selected as anodes to source current (the plus signs). E2 has been designated in electrode parameter interface 86 to receive a relative strength of 60% of the amplitude (current) specified in the waveform parameter interface 84 (i.e., +0.6 A), while E5 will receive the remaining 40% (+0.4 A). Because there is only one cathode E4, that electrode will receive 100% of the sunk current (−A). The relative anode and cathode percentages or strengths are also displayed in the leads interface 92 along with their polarities (+or −) (96). Also provided in the electrode parameter interface 86 are buttons to allow the various anode electrodes and cathode electrodes to be equalized to 100%. Other means for displaying the selected electrodes, their polarities, and their relative strengths are possible, including the use of different colors, actual numerical values for the amplitudes rather than percentages, etc.

Stimulation parameters interface 82 includes a mode menu 90 to allow the clinician to choose different modes for determining stimulation parameters. (More on this later, but by way of preview, FIG. 5 largely shows manual stimulation parameter determination options). An advanced menu 88 is also provided to allow for the setting of other variables relevant to the stimulation waveform produced (again, as discussed later).

FIG. 6 shows an example of the waveforms as specified by the stimulation program defined in CP GUI 64 of FIG. 5, which stimulation program can be telemetered to the patient's IPG 10 and stored in the CP computer 51 of the CP system 50.

It should be noted that the CP GUI 64 as depicted allows for the definition of uniphasic pulses—i.e., pulses with a single phase 97 (of amplitude A and duration D) that is either anodic or cathodic. However, such uniphasic-defined pulses may actually be applied by the IPG 10 as one phase of a biphasic pulse 99. This is shown in the dotted lines of FIG. 6, in which each defined pulse phase 97 (+/−A, D) is followed by a second pulse phase 98 of opposite polarity (−/+A′, D′). As is known in the art, provision of the second pulse phase 98 actively recovers charge build up on capacitances inherent in the system (such as DC blocking capacitors in the electrode current paths, capacitances inherent in the electrode (tissue interface, or capacitances inherent in the tissue itself) after provision of the first phase 97. As is also known, the phases 97 and 98 of the bi-phasic pulse 99 can have different amplitudes (A, −A′), with the first amplitude A having a therapeutic effect, but with the second amplitude −A′ being sub-threshold from the standpoint of neurostimulation. Likewise, the durations of the phases 97 and 98 (D, D′) can be different, and are typically determined with reference to the amplitudes A and −A′ to define the same amount of charge in each phase (i.e., A*D=A′*D′).

Further, passive charge recovery 100 can be applied in the IPG 10 to recover any remaining charge build up after pulsing by shorting the (active) electrodes 24 to a common potential. Such passive recovery 100 can occur either after the first pulse phase 97 (if a second active-charge-recovery phase 98 is not used), or after the second pulse phase 98.

If such additional pulse phases 98 and/or 100 are to be used in the IPG 10, they can be defined and programmed in different manners. First, although not shown, the CP GUI 64 of FIG. 5 could include additional “advanced” options for defining the timing and duration (and in the case of second pulse phase 98, its amplitude −A′) of such additional phases 98 and 100 relative to the first pulse phases 97. Alternatively, the CP GUI 64 may operate to define such pulse phases 98 and 100 automatically in accordance with programmed rules and without clinician involvement. In either case, such additional pulse phase details may be stored in the CP computer 51 of the CP system 50 as part of the stimulation program along with the primary therapeutic parameters (e.g., A, D, F, Ex, Px). Alternatively, automatic definition and programming of such additional pulses phases 98 and 100 may occur in the IPG 10 upon receipt of the stimulation parameters for pulse 97, again using programmed rules.

### BRIEF DESCRIPTION OF THE DRAWINGS

FIG. 1 shows an Implantable Pulse Generator (IPG) useable for Deep Brain Stimulation (DBS) for example in accordance with the prior art.

FIGS. 2A and 2B show cross-sections of IPGs respectively having coil and RF communication antennas in accordance with the prior art.

FIG. 3 shows the implantation of the IPG of FIG. 1 in an IPG DBS patient, in accordance with the priori art.

FIG. 4 shows a clinician programmer (CP) system including a CP computer having CP software for allowing a clinician to determine optimal stimulation parameters for an IPG patient in accordance with the prior art.

FIG. 5 shows a portion of the Graphical User Interface (GUI) rendered by the CP software on the CP computer's display, specifically a portion for manual setting stimulation parameters, in accordance with the prior art.

FIG. 6 shows the waveforms delivered to various electrodes as specified in the GUI of FIG. 5 in accordance with the prior art.

FIG. 7 shows an improved CP system including an Optical Head-Mounted Display (OHMD), in accordance with an embodiment of the invention.

FIG. 8 shows various communication links that can be established in the improved CP system of FIG. 7 in accordance with an embodiment of the invention.

FIG. 9 shows various modules in the CP software in accordance with the prior art.

FIG. 10 shows simplistic rendering of certain of the software modules of FIG. 9 in the GUI of the OHMD in accordance with an embodiment of the invention.

### DETAILED DESCRIPTION

The inventors have noticed that use of the CP system 50 and its software 66 as described earlier can be a distraction to the clinician, particularly when the clinician is using the system to determine optimal stimulation programs for a patient having a Deep Brain Stimulation (DBS) IPG 10.

When a clinician experiments by using different stimulation parameters (A, D, F, Ex, Px) on a non-DBS IPG patient—for example, a patient who has a Spinal Cord Stimulator (SCS) IPG 10 for the treatment of chronic back pain—the clinician may be wholly reliant on the patient to inform the clinician concerning the effects of any parameter changes the clinician is making at the CP 50, because the clinician cannot observe the effect of such changes. Instead, such feedback concerning the efficacy of stimulation parameters changes is normally provided verbally by the patient (“I feel less pain”; “I feel tingling”; “that's a little uncomfortable”; etc.).

Moreover, in an SCS context, the physiology involved suggests that it may take a moment (e.g., a few seconds) for new stimulation parameters to render a noticeable effect in the patient. As such, it is less problematic in this context that the clinician may be pre-occupied by the CP system 50: the clinician can make stimulation parameter changes via CP GUI 64; send them to the IPG 10; listen for patient feedback within a reasonable timeframe (a few seconds); and make subsequent logical changes to the parameters as necessary to try and arrive at optimal stimulation programs. Stated simply, it is not strictly necessary for the clinician to observe the SCS IPG patient as stimulation parameters are changed via CP GUI 64.

By contrast, the clinician is less able to rely on patient feedback when determining the effectiveness of changes in stimulation parameters in a DBS IPG patient. This is in part because changes in a DBS IPG patient's symptoms (e.g., tremors; walking ability; hand stability and dexterity) may not be discernable or quantifiable by the patient, and so the DBS IPG patient may have little or nothing useful to say to the clinician by way of feedback.

Instead, the effectiveness of stimulation parameter changes in a DBS IPG is dependent in significant part on the clinician's observation of the patient's symptoms. Moreover, given the difference physiologies involved in SCS and DBS, symptomatic changes may be essentially immediately observable upon changing stimulation parameters. It is therefore more important in the inventors' view for a clinician when discerning optimal stimulation programs to view the DBS IPG patient as changes to her stimulation parameters are made in the CP GUI 64. The complicated nature of the CP GUI 64 though requires clinician focus, and so immediacy of the effectiveness of stimulation parameter changes can be missed as the clinician is repeatedly required to glance between the CP GUI 64 and the patient with each stimulation parameter change.

Accordingly, the inventors disclose herein an improved CP system including improved CP software and an optical head-mounted display (OHMD), such as glasses or goggles, which a clinician can use to adjust a patient's stimulation parameters to arrive at stimulation programs that are optimal for the patient, and to receive status information from the implant. The OHMD is preferably wirelessly coupled to bi-directionally communicate with the CP computer in the improved CP system, and is enabled by the improved CP software to render an OHMD GUI on the OHMD to allow the clinician to program the patient's IPG.

The OHMD receives from the CP software certain data to enable certain functions that may traditionally have been enabled in the GUI of the CP computer (CP GUI). The OHMD is preferably rendered in a simple format in the OHMD and within the clinician's field of view. OHMD GUI functionality in the CP system is preferably limited to critical functionality as described further below, and may include at least functionality for changing one or more of the stimulation parameters (A, D, F, Ex, and Px) discussed earlier. All other non-critical functionality of the CP software can be rendered in the CP GUI of the CP computer in traditional fashion, and as described in the Background. When the clinician interacts with the OHMD GUI to make changes to the patient's stimulation parameters, the OHMD can either transmit the changes to the patient's IPG as a command either directly or indirectly via the CP computer. In any event, the clinician can beneficially make such therapy changes while continuing to observe the patient, which is particularly useful in the context of a DBS IPG patient for the reasons discussed.

Optical head-mounted displays (OHMDs) have been used in clinical settings to allow a clinician to simultaneously view a patient while also reviewing supplemental information of interest to the patient's treatment, which information may be fed to the OHMD by a cooperative system. For example, an OHMD coupled to an operating-room computer device can receive and display for the surgeon's immediate convenience information concerning a patient's vital signs. A more complicated example of an OHMD useable in the medical context allows a surgeon to simultaneously view a patient and her medical imaging data (e.g., X-ray, MRI, ultrasound, endoscope, etc.), which imaging data may be taken in real time. Such imaging data may be superimposed on the patient in the correct position in more sophisticated system.

OHMDs come in several different forms. In one form, an OHMD comprise an opaque screen that is held proximate to the wearer's eyes, and may include separate left and right screens for each eye. As the screens are opaque, this type of OHMD may include a forward-facing camera to capture “real world” image data that can be merged with other supplemental information provided by the computing system coupled to the OHMD. Other types of OHMD are transparent or semi-transparent, and allow the wearer to see the world around them, but superimpose supplemental information on the wearer's field of vision. However, traditional OHMDs may lack the ability to receive a clinician's input, and more specifically may lack input means for the clinician to control therapy being provided to the patient.

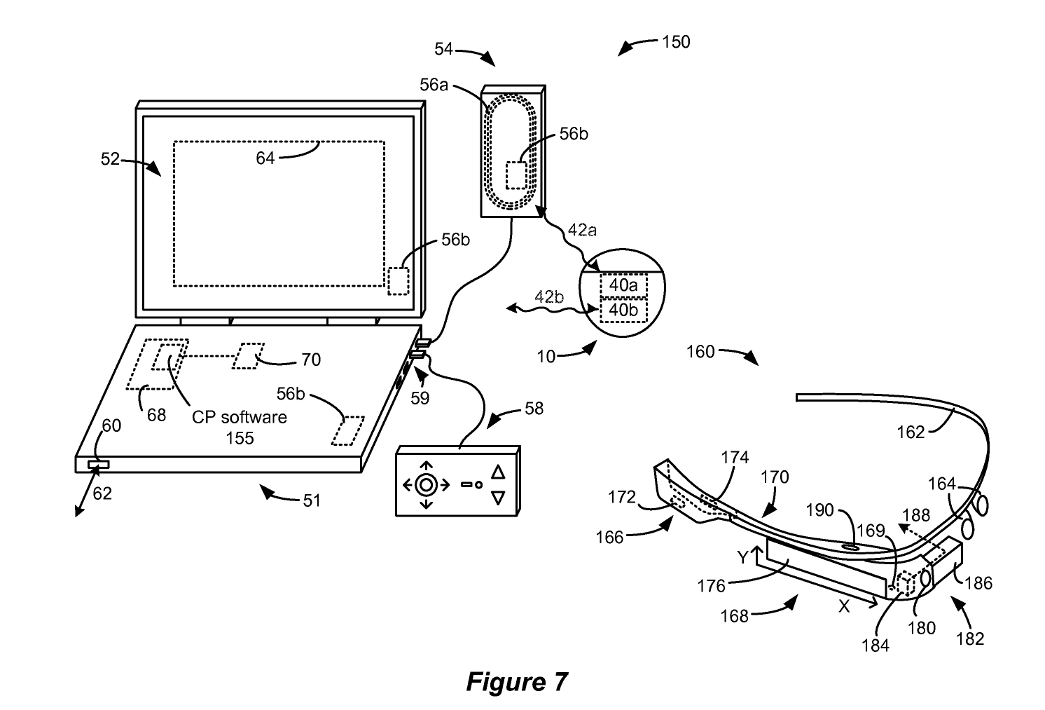

Recently, OHMDs with improved input capabilities have been introduced which the inventors consider suitable for use in an improved clinician programmer (CP) system 150, as shown in FIG. 7. As a comparison with FIG. 4 shows, the improved CP system 150 can retain much or all of the aspects of the prior-art CP system 50. New to the system 150 is an OHMD 160, which can comprise for example the Google Glass™ OHMD, developed by Google, Inc. of Mountain View, Calif., and as described in US Patent Application Publications 2013/0044042; 2013/0070338; and 2013/0293580. However, any other OHMD currently existing or later developed could also be used in the improved CP system 150 so long as it is capable of providing the functionality described herein. A Google Glass OHMD 160 is depicted and discussed with particularity, but should not be understood as limiting the scope of the invention. Also new to the CP system 150 is improved CP software 155 designed to operate with both the CP computer 51 and the OHMD 160, as described further below.

As shown, OHMD 160 is configured to be wearable much like a pair of standard eyeglasses, and includes a frame 162 which also serves as the temples supported by the wearer's ears, and nose pads 164. Lenses (e.g., corrective or sunglasses lenses) may be affixed to the frame 162, but are not shown in FIG. 7. OHMD 160 may also be worn in conjunction with a wearer's normal eyeglasses.

Plastic affixed to the frame 162 generally defines a rearward housing 166 and a forward housing 168 on the OHMD 160's right temple. Plastic also defines a pass-through portion 170, which as well as defining a space for the wearer's right ear, also provides for the passing of wires between the two housings 166 and 168. The rearward housing 166 holds a rechargeable battery (not shown), and includes a micro USB port 172 on its underside which can be used for wired communications or to recharge the battery via a wall outlet. A bone-conduction audio transducer 174 in the rearward housing 166 protrudes through the plastic and presses over the right ear to permit the wearer to hear sounds provided by the OHMD's interface, which is explained below. OHMD 160 could also include a more-traditional audio speaker as well.

The forward housing 168 includes a printed circuit board (not shown), which supports the OHMD 160's main electronics, such as its microprocessor, and movement sensors providing input to a motion detector module in the electronics, including a three-axis accelerometer, a three-axis gyroscope. A three-axis magnetometer is also provided, and operable as a compass for example. Also included in the forward housing 168 is a touch sensor (not shown), which allows the outer surface of the forward housing to operate as a touch pad 176. The touch pad 176 is sensitive to the wearer's touch across the two-dimensional expanse (X and Y) of the outer surface of the foreword housing 168, and can additionally be pressed (“tapped”) similar to a button. The underside of the forward housing 168 additionally includes a microphone 169 for the receipt of voice input (not shown) in addition to inputs receivable by the touch pad 176. The electronics of the OHMD 160 will include a voice detection module for interpretation of spoken voice inputs, as is well known. Forward housing 168 also includes a depressible on/off button 190. If the OHMD 160 is merely in a sleep mode rather than off, it can be “awoken” by tapping the touch pad 176, by the user tilting her head back (which motion is detectable via the accelerometers and/or gyroscopes), or by speaking a wake-up instruction (such as “OK Glass”) for example.

A front facing portion of the forward housing 168 includes a forward-facing camera 180 for taking pictures and video, and further includes a display portion 182 of the OHMD 160. Details concerning the display portion 182 are discussed further in U.S. Patent Application Publication 2013/0070338, which is incorporated herein by reference. Without going into detail, the display portion 182 includes an LED array 184 powered by the OHMD's microprocessor. Images 188 created at the LED array 184 are directed to a prism 186 containing a polarizing beam-splitter that direct the images 188 to the wearer's right eye. In this manner, the user is able to perceive the images 188 generated by the OHMD 160 and output by the display portion 182, which images 188 are provided slightly to the right of the wearer's center of vision, thus allowing the wearer to see the real world and the images on the display portion 182 simultaneously.

OHMD 160 in this example further includes bi-directional short-range RF communication capabilities, and preferably includes hardware and software compliant with Bluetooth and Wi-Fi communication standards, such as were discussed in the Background. Such wireless communication capabilities of the OHMD 160 provide for improved connectivity in the CP system 150, and various wired and wireless connections are shown in the network of FIG. 8. The OHMD 160 can couple by wire to the CP computer 51 in the CP system 150 (e.g., at USB ports 172 and 59 respectively), although because CP computer 51 would typically support either or both of Bluetooth and WiFi, the link to the OHMD 160 is preferably wireless using one of these standards which improves the freedom of the clinician.

How the OHMD 160 ultimately communicates with the IPG 10 to change its stimulation parameters or to receive IPG 10 status information depends on the type of antenna present in the IPG 10, and as before either a magnetic inductive link 42a or a short-range RF link 42b (see FIGS. 2A, 2B, and 4) can be used. If the IPG 10 includes a coil antenna 40a (FIG. 2A), communications from the OHMD 160 are preferably wirelessly routed to the CP computer 51, then to the wand 54 by its cable, and then wirelessly to the IPG 10 via a magnetic inductive link 42a. This is necessary because OHMD 160 as described lacks an antenna coil compliant to communicate with the IPG's coil 40a via a magnetic inductive link 42a. If however the IPG 10 includes a short-range RF antenna 40b (FIG. 2B), the OHMD 160 may be able to wirelessly communicate with the IPG 10 directly along a short-range RF link 42b, particularly if the short-range RF antenna 40b used in the IPG 10 is complaint with the short-range RF standard (e.g., Bluetooth and WiFi) supported by the OHMD 160. If the IPG antenna 40b is not compliant with these standards, then indirect communication from the OHMD 160 to the IPG 10 via the CP computer 51 may once again be necessary, with the CP computer 51 using one of its IPG-compliant short-range RF antennas 56b (either in the device 51 or in the wand 54; see FIG. 4). As communication with the OHMD 160 is bi-directional, receipt of status information from the IPG 10 at the OHMD 160 can also occur along these direct or indirect routes.

The OHMD 160 is further able to communicate with networks 194 such as the Internet. Such communication can occur indirectly through the CP computer 51, with the OHMD 160 being either wired (172/59) or wirelessly connected to the CP computer 51, and with the CP computer 51 being either wired (60) or wirelessly connected to the network 194. The OHMD 160 may also more directly connect to the network 194 via a Bluetooth or WiFi gateway 192. Such wireless gateways 192 can comprise traditional wireless network “hot spots,” and also can comprise other devices that can communicate with both the network 194 and the OHMD 160. For example, a mobile device 200, such as a smart phone or tablet (which may also operate as the patient external controller 44b) is generally both Bluetooth and Wi-Fi compliant, and is further able to reach the network 194 via a cellular network 202. Therefore, the OHMD 160 can reach the network 194 (Internet) through this route.

The ability of the OHMD 160 to reach the Internet provides expanded options regarding patient therapy in CP system 150. For example, a clinician may via the Internet at a remote computer 204 both view the patient and change the patient's stimulation parameters just as if she were in the room with the patient. For example, another clinician or assistant can be with the patient, wear the OHMD 160, and couple it to the CP system 150. The remote clinician can log in to the CP system 150, and see the patient—for example, via the OHMD 160's camera 180. The remote clinician can also adjust stimulation parameters, either by using the otherwise standard CP GUI 64 provided on the CP computer 51, or using a simplified OHMD GUI otherwise provided to the OHMD 160, as explained shortly, which the CP computer 51 can serve to the remote clinician's computer 204 as well as to the OHMD 160.

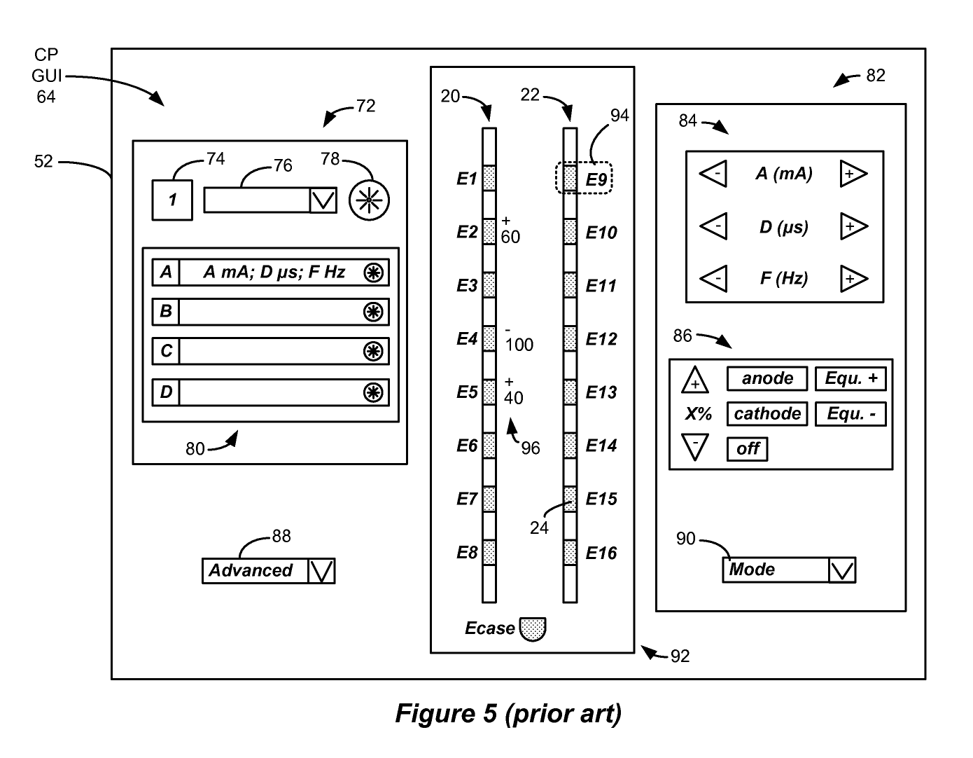

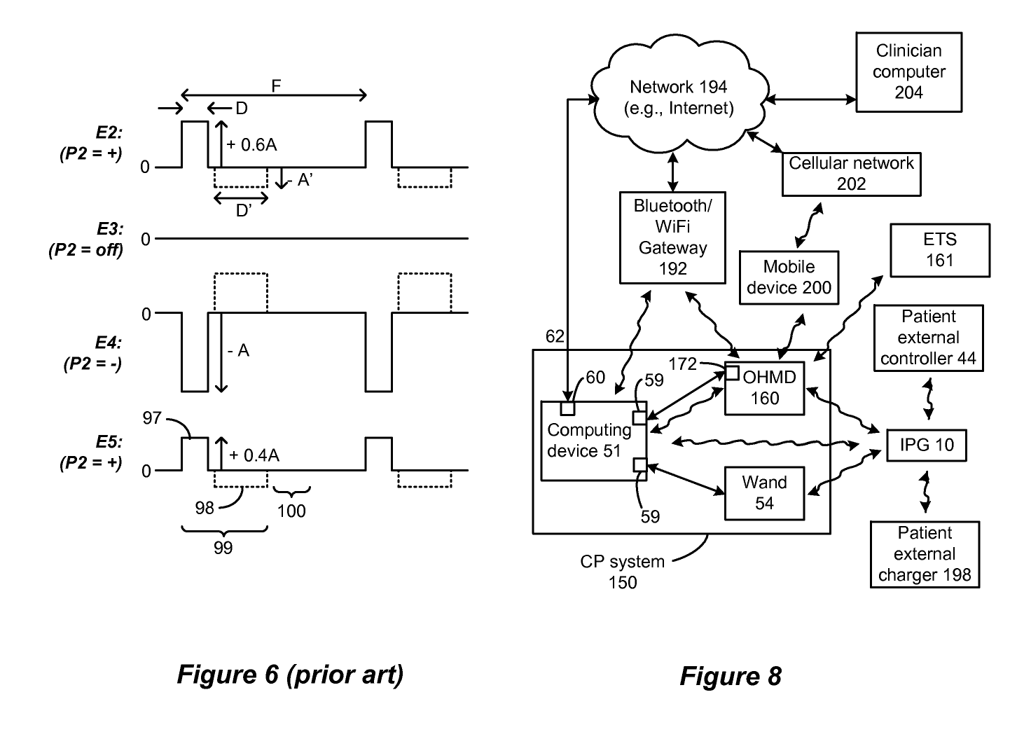

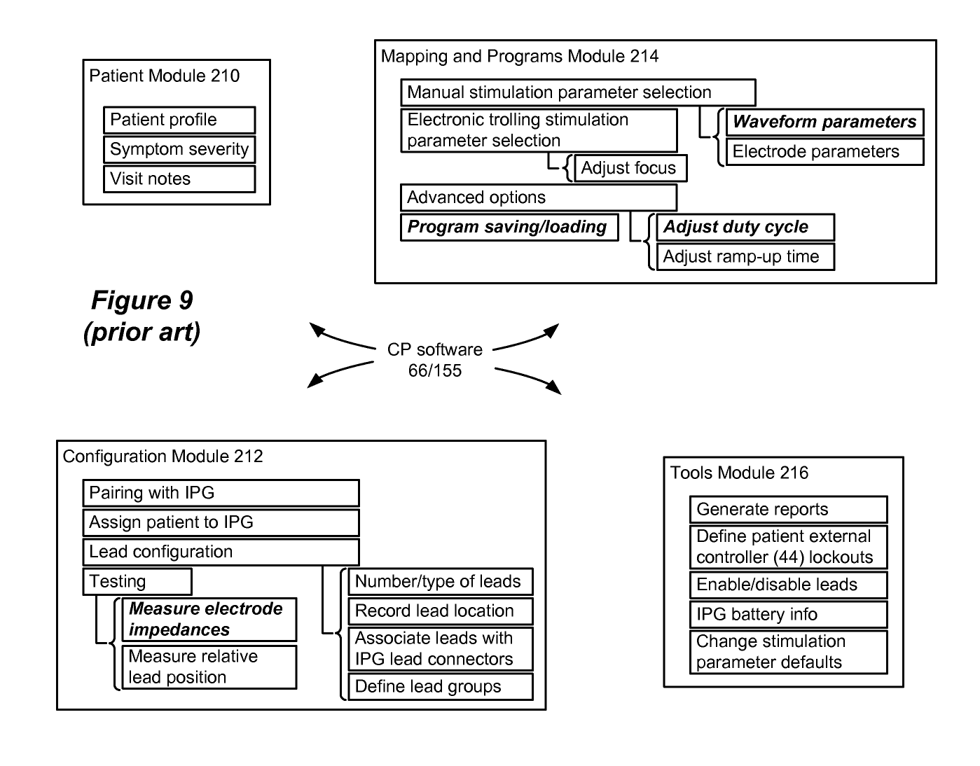

An issue to consider when using an OHMD 160 in CP system 150 is the relatively complexity of the CP software and the CP GUI 64 that it can render, while the user interface of the OHMD 160 is relatively simple by comparison. FIG. 9 illustrates further functional aspects of the prior art CP software 66 beyond that described earlier (see FIG. 5), which functionality may also be present in improved CP software 155. As shown, the CP software 66/155 is generally organized into software modules, each presenting its own (sub) modules depending on the options chosen by the clinician. Of course, although not illustrated, the functionality of each of these modules may change the CP GUI 64 rendered on the CP computer 51 as they are activated.

A first module usually accessed by the clinician in the CP software 66/155 is a patient module 210, which allows basic information about the patient (name, age, address, etc.); the relative severity of his symptoms (including when stimulation is on or off); and other random visit notes to be stored in a patient record.

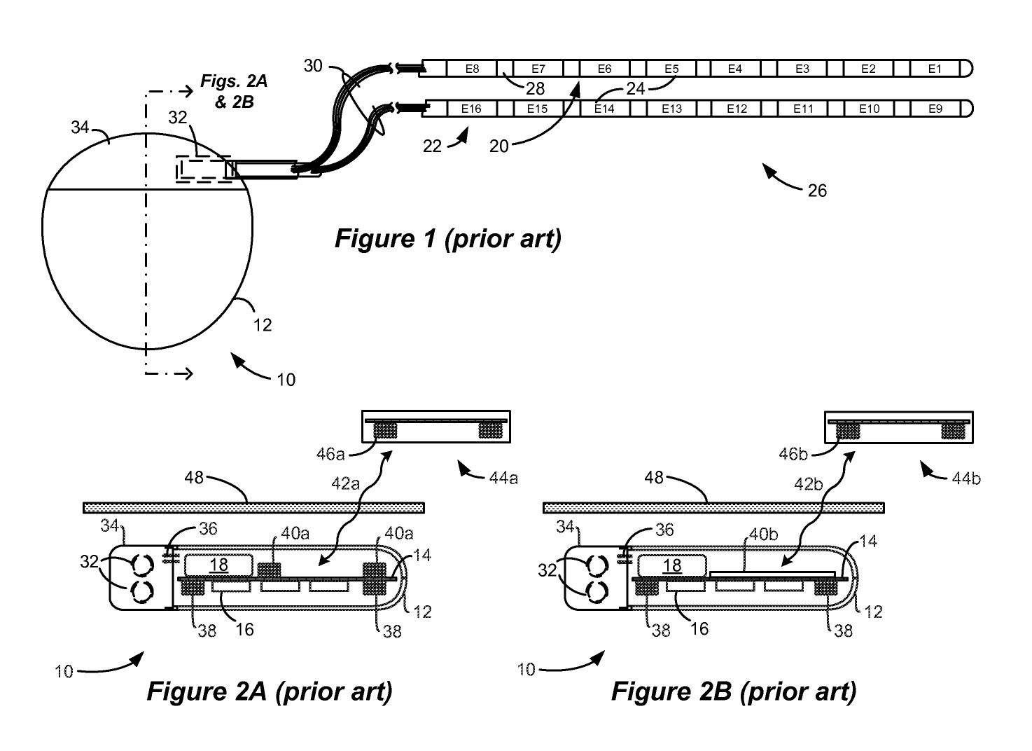

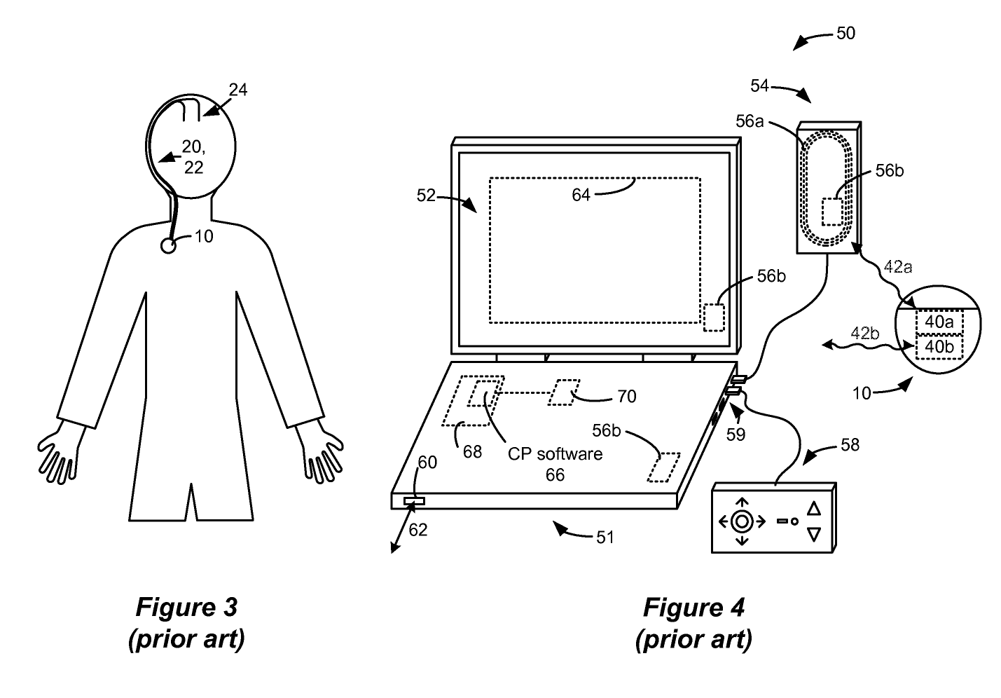

A configuration module 212 allows for pairing the CP system 50/150 to the patient's IPG 10 so that communications between the two—including eventually the transmission of new stimulation parameters—can begin. Thereafter, the IPG 10 in communication with the CP system 50/150 can be assigned with the patient. A lead configuration module allows for the entry of the type and number of leads (e.g., 20 and 22) used with the patient's IPG; allows for the relative location of the leads with respect to the patient's anatomy to be recorded; allows the leads to be associated with particular lead connectors 28 (FIG. 1) on the IPG 10; and allows various leads that should be programmed together to be defined in a lead group. The configuration module 212 further allows various tests to be run, such as measuring the impedance of the electrodes 24 (e.g., to determine faulty open- or short-circuited electrodes); and to determine the relative positioning between leads in the same lead group.

Certain aspects of the mapping and programs module 214 were discussed earlier (FIG. 5), but that discussion essentially focused on operation of the manual stimulation parameter selection module, which rendered and received input from the waveform parameter interface 84 and the electrode parameter interface 86 of the stimulation parameters interface 82. Other modules not illustrated earlier can also be used to select stimulation parameters (see, e.g., mode menu 90; FIG. 5), such as an electronic trolling module, which comprises an automated programming mode that performs current steering along the electrode array by moving the cathode in a bipolar fashion. Recommended stimulation parameters are calculated by this trolling module using a mathematical model of field potentials based on average values of tissue (e.g. CSF) thickness and resistivity. The joystick 58, or directional arrows in the CP GUI 64 active when this module is chosen (not shown) can be used to steer a central stimulation point up and down, and left and right, along the leads 20 and 22. Further advanced options in this electronic trolling module (see e.g., mode menu 90; FIG. 5) can be used to adjust the distance or “focus” between the anodes and cathodes.

Still other advanced options in the mapping and programs module 214 (see, e.g., advanced menu 88; FIG. 5) allow for the setting of a duty cycle (on/off time) for the stimulation pulses, and a ramp-up time over which stimulation reaches its programmed amplitude (A). The mapping and program module 214 allow allows for different stimulation programs to be saved and loaded for the patient, as explained earlier.

The CP software 66/155 further provides a tools module 216 providing various modules providing options to generate various reports; to prevent certain stimulation parameters from being changeable by the patient using his external controller 44 (FIGS. 2A and 2B); to enable or disable the leads 20 or 22; to view information concerning the IPG's battery 18 (FIGS. 2A and 2B); and to change default values and increments for the stimulation parameters.

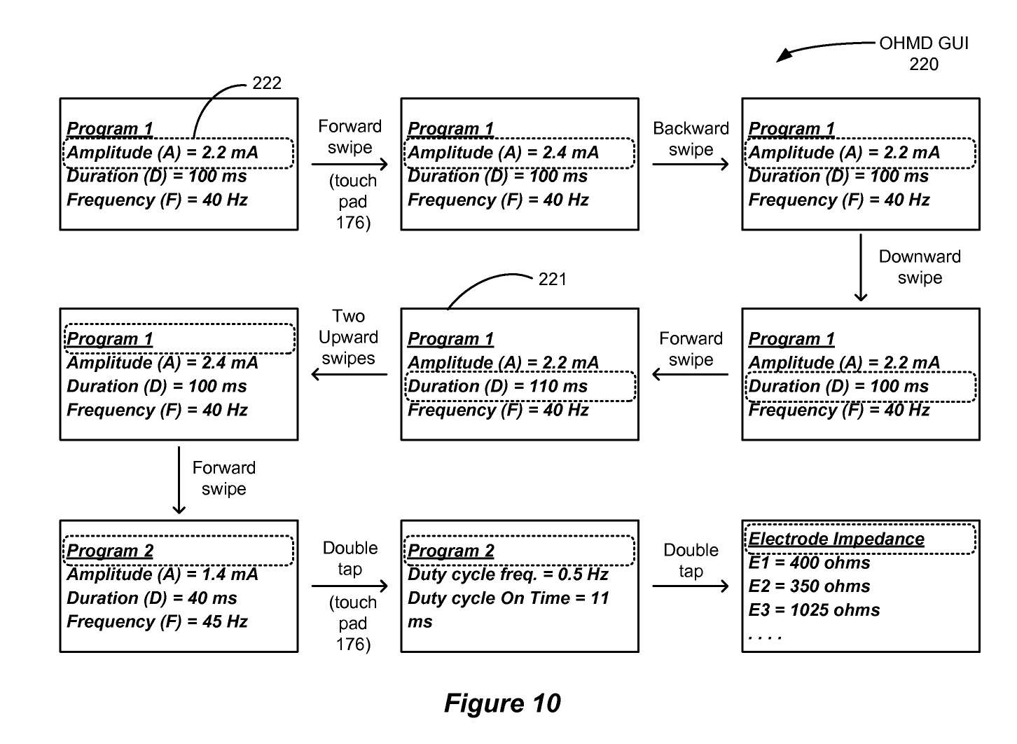

While FIG. 9 provides a sense of the full complexity of the functionality of the various modules in CP software 66/155, the inventors realize that the comparatively simple user interface provided by the OHMD 160 may not easily handle that full functionality (although it could). Thus, the improved CP software 155 in the CP computer 51 controls the OHMD 160 to render an OHMD GUI 220 (FIG. 10) that provides only limited clinician programmer functionality; other CP functionality remains in the CP computer 51 and is accessible by the clinician through its CP GUI 64.

As discussed subsequently, the CP software 155 preferably enables the limited functionality in the OHMD GUI 220 in a simple, non-distracting manner to the clinician while allowing the clinician to simultaneously observe symptomatic changes in the patient. As such, the OHMD GUI 220 may be limited to CP functionality that is critical. Which clinician programmer functions are sufficiently “critical” to warrant enabling in the OHMD GUI 220 as opposed to in the CP GUI 64 of the CP computer 51 will be a matter of preference, and may depend on the nature of the therapy the IPG (or more broadly, the IMD) provides. Critical functionality may comprise IPG programming functions, such as those that are uniquely implicated when the clinician is determining optimal stimulation parameters for the patient, and may comprise at least the ability to use the OHMD GUI 220 to adjust the stimulation parameters (A, D, F, Ex, Px) discussed earlier. Critical functionality may also comprise certain IPG monitoring functions in which IPG status information is provided to the OHMD GUI 220, such as the current values of the stimulation parameters, IPG battery status, electrode impedances, etc. Critical functionality may also be those functions that the clinician considers important to access via the OHMD 160 when working with a patient, whether determining optimal stimulation parameters or otherwise. In this regard, the improved CP software 155 may alternatively allow a clinician to select which CP functionality should be render at the OHMD GUI 220. In short, “critical” may simply comprise a subset of the functionality traditionally provided by desktop-based CP software 66, as described in the Background, and doesn't not necessarily imply importance from a safety or therapeutic standpoint.

Portions of the functionality of CP software 155 that are not made accessible to the clinician in the OHMD GUI 220 are still preferably accessible to the clinician via the CP GUI 64 provided on the CP computer 51 as just noted. Therefore, a clinician using the improved CP system 150 may access both non-critical functionality in the CP GUI 64 of the CP computer 51, and critical functionality in the OHMD GUI 220 of the OHMD 160.

Functionality enabled by the CP software 155 in the OHMD GUI 220 of the OHMD 160 may be redundantly enabled in the CP GUI 64 of the CP computer 51, therefore allowing the clinician to interface with either to access the functionality. In another example, functionality enabled by the CP software 155 in the OHMD GUI 220 of the OHMD 160 may be lacking in the CP GUI 64 of the CP computer 51, and hence only accessible through the OHMD 160.

Even if CP software 155 is programmed to enable particular CP functionality in the OHMD 160, the CP software 155 may be programmed to first verify that the OHMD 160 is in fact registered with and able to communicate with the CP system 150. If so, the CP software 155 can enable such functionality via the OHMD GUI 220 (either exclusively of, or redundantly with, the CP GUI 64 as just discussed). If the OHMD 160 is not recognized by the CP software 155, it may instead instruct the CP GUI 64 on the CP computer 51 to render such functionality instead. If the CP software 155 later verifies the OHMD 160, the CP software 155 may enable such functionality at the OHMD 160 via OHMD GUI 220 at that later time.

While the CP functionality enabled via the OHMD GUI 220 is described in the below example with reference to functions provided by CP software of the prior art for simplicity and to ease understanding, it should be understood that the OHMD 160 can also enable new or later-developed functionality in the CP system 150.

FIG. 10 show an example of the OHMD GUI 220 provided by the CP software 155 to a clinician wearing the OHMD 160 (i.e., through its display portion 182; FIG. 7), and shows examples of how the clinician can navigate the OHMD GUI 220. Consistent with the above explanation, the CP functionality enabled at the OHMD 160 via OHMD GUI 220 is limited. Specifically, the functionality enabled in the OHMD GUI 220 in the example of FIG. 10 is limited to bolded software modules in FIG. 9, and thus allows the clinician to review and adjust the waveform parameters (A, D, F; see interface 84; FIG. 5); to review and adjust the duty of cycle of stimulation (see advanced menu 88; FIG. 5); to review electrode impedances (a functional module in configuration module 212; FIG. 9); and to load and save programs (see interface 72; FIG. 5).

The OHMD GUI 220 rendered in this example of FIG. 10 is shown as a series of cards 221. In this example, the clinician can only view one card at a time, but may navigate between cards and enter new stimulation parameter values using the touch pad 176 on the OHMD 160. (Voice inputs and user gestures can also be used for navigation and data entry as explained further below).

The first card 221 in the OHMD GUI 220 illustrates and allows control of the waveform parameters (A, D, and F) for the patient's Program 1. This first card may be the first presented to the clinician via the OHMD GUI 220, or may be a card that is later “swiped” to using the touch pad 176, such as from an initial home screen of the OHMD GUI 220. Shown in this first card is a cursor 222, which at present highlights the amplitude parameter currently stored for program 1.

In this example, the cursor 222 is moved by swiping up and down on the touch pad 176, while parameter values are increased or decreased by swiping forward or backward on the touch pad 176. Upon review of the first card 221, the clinician wishes to increase the amplitude for Program 1, which is already highlighted by the cursor 22 and currently set to 2.2 mA. Thus, the clinician swipes forward on the touch pad 176 to increase this value by a set amount or increment, and so is now adjusted to 2.4 mA. Such changes implemented at the OHMD 160 are sent immediately to the IPG 10 as a command, perhaps via the CP computer 51 as described earlier, and are also sent to the CP software 155 for storage in Program 1 in the CP computer 51. The next card 221 shows the result of a backward swipe, which decreases the amplitude value back to 2.2 mA.

A downward swipe moves the cursor 222 to the duration parameter, which is currently set to 100 ms, but which can also be similarly adjusted. The forward swipe shown thus increases its value to 110 ms which new value is sent to the IPG 10. Two upward swipes at this point places the cursor 222 on the program, which too can be changed. As shown, a forward swipe brings up the waveform parameters for Program 2, which new parameters would also be sent to the patient's IPG 10.

A tapping action can also be used to provide different navigation or control capabilities in the OHMD GUI 220. In the example shown a “double tap”—two quick successive taps—changes the parameters for the current program, which the clinician can view, and change. The first double tap as shown in FIG. 10 allows the clinician to change an advanced stimulation setting for the current program, namely the duty cycle. After that parameter is changed (not shown), another double tap might allow the clinician to review parameters that don't involve adjustment to a patient's stimulation. As shown in FIG. 10, the example of measuring and displaying the electrode impedances is shown.

Again, FIG. 10 provides a simple non-limiting example of an OHMD GUI 220. The data displayed and controlled, the manner in which it is presented, the organization of the data on cards 221 or other GUI structures, the manners of selection and control of the OHMD GUI 220 at the OHMD 160, can be changed to suit the environment at hand.

If the OHMD 160 comprises the Google Glass device, the development of such cards 221 as shown in OHMD GUI 220 is facilitated by the Google Glass Developers Kit, which is available at https://developers.google.com/glass/. Essentially, such developer kits allow one skilled in the art to take functionality from the CP software of the CP system, and convert it to the OHMD GUI 220 format shown in FIG. 10. Typically, an XML programming language is used to program GUI cards 221 which can receive input from the touchpad 176, by voice input, by user gestures, etc.

According to some embodiments, the OHMD GUI 220 is compiled by the CP software 155 and stored in the OHMD 160. Alternatively, the relevant aspects of the OHMD GUI 220 may be sent from the CP software 155 in the CP computer 51 to the OHMD 160 as required, i.e., when the clinician interacts with the OHMD GUI 220.

It should be remembered that input interface of the OHMD GUI 220 is preferably not limited to touch inputs such as enabled by the touch surface of the touch pad 176. One or more buttons on the OHMD 160 may be used as well both for OHMD GUI 220 navigation and for data entry or adjustment. Additionally, navigation and data entry and adjustment can also be spoken by the user and received by the OHMD 160's microphone 169, and processed by its voice detection module. Voice input may result in the OHMD GUI 220 forming a command to be transmitted to the IPG 10. For example, the clinician upon reviewing the first card 221 in FIG. 10 may change the amplitude by speaking “OK, Glass. Increase amplitude,” or “Amplitude equals 2.4,” which command can be transmitted to the IPG 10 for action as discussed previously. The clinician may also navigate the OHMD GUI 220 using voice inputs, such as by speaking “next value,” next card,” “program 2,” etc.

The motion detectors in the OHMD 160 (accelerometers and/or gyroscopes) additionally allow for input via user gestures. For example, instead of swiping right and left, or up and down on the touchpad 176 to navigate or enter data, user input could similarly be effected by the user turning his head to the right or left, or up and down.

Nor preferably is the OHMD GUI 220 limited to providing viewable graphical outputs (using the display portion 182 and LED array 184 for example). Other user-discernable outputs can be audibly rendered as part of the OHMD GUI 220 using the OHMD 160's audio transducer 174 or speaker. For example, the clinician might instruct (by touch, voice, or gesture) the OHMD 160 to provide an audible summary of the stimulation parameters, which may prompt the OHMD GUI 220 to audibly broadcast “Amplitude equals 2.2; duration equals 100; frequency equals 40; cathodes equal E6 and E7; anodes equal E8.” Such audibly-rendered information is particularly useful if the information is not presently being display by the OHMD GUI 220, on a card 221 for instance. A vibratory motor or other tactile means of output can also be used in the OHMD 160.

As with graphically-displayed information, audible presentation to the user can also include status information transmitted from the IPG 10. For example, a clinician viewing the cards 221 in the OHMD GUI 220, and perhaps suspecting a problem, may speak “OK, Glass. Electrode impedance test.” The OHMD GUI 220 upon receiving this instruction can transmit it to the IPG 10, which will run the test, and transmit the electrode impedances values back to the OHMD GUI 220. Upon receiving the values, the OHMD GUI 220 may audibly state for example “Electrode impedances within limits” to quickly inform the clinician of this status without requiring the clinician to access and digest the particular values visually—for example on the last card as shown in FIG. 10.

The inventors consider the OHMD GUI 220 to be simpler and less distracting for the clinician when changing an IPG patient's stimulation parameters, and is further beneficial in allowing the clinician to view the patient at the same time that such changes are made at the OHMD 160. As noted, such immediate observance of a DBS IPG patient to simulation parameters changes can be especially insightful to the clinician in determining optimal stimulation programs comprising such parameters.

Although the improved clinician programmer system has been disclosed as useful in the context of a DBS IPG patient, the system is not so limited, and may also be used with patients having other types of IPGs or implantable medical devices (IMD) more generally. For example, it may be useful for a clinician to observe a patient having a sacral nerve stimulator (SNS) for the treatment of various urinary ailments such as urinary urge incontinence, urinary frequency, and urinary retention. In this setting, a clinician adjusting stimulation parameters for the SNS patient may wish to immediately look for visual cues as stimulation parameters are changed, such as toe twitching, which may inform the clinician that the stimulation is too intense for the patient, or that the wrong nerves are being recruited and thus that the electrodes chosen for stimulation should be changed to other locations on the lead. In another example, the improved clinician programmer system may be used to adjust the parameters of a patient having a Spinal Cord Stimulator (SCS) IPG for the relief of chronic low back pain.

Further, the disclosed OHMD 160 and its OHMD GUI 220 may be useful in controlling and monitoring the operation of a more generic medical device, which medical device need not be implanted within a patient. For example, the OHMD 160 and its GUI 220 may be used to control an External Trial Stimulator (ETS) 161, as shown in FIG. 8. As described in U.S. Patent Application Publication 2014/0358194, which is incorporated herein by reference in its entirety, an ETS 161 can be used to mimic operation of an IPG 10 during a trial period before an IPG 10 is implanted but while the IPG leads 20 and/or 22 (FIG. 1) are implanted in the patient. The ETS 161 during such a trial period is typically carried or worn by the patient, and couples to leads 20 and/or 22 (FIG. 1) via lead extension passing through the patient's skin (not shown). OHMD 160 and OHMD 220 can also be used to control other external pulse generators, such as transcutaneous pulses generators (e.g., transcutaneous electrical nerve stimulators (TENS)), or other external medical devices more generally.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for adjusting the stimulation parameters of a patient having a pulse generator medical device, comprising:
a clinician programmer (CP) computer having clinician programming (CP) software;
an optical head mounted display (OHMD) coupled to the CP computer by a communication link;
wherein the CP software is configured to cause the OHMD to render a graphical user interface (GUI) at the OHMD that is accessible by the clinician to adjust at least one stimulation parameter of the patient's medical device.

2. The system of claim 1, wherein the communication link comprises a wireless link between the OHMD and the CP computer.

3. The system of claim 1, wherein the GUI at the OHMD is configured to generate a command for the patient's medical device when the at least one stimulation parameter is adjusted.

4. The system of claim 1, wherein the OHMD further comprises an antenna configured to wirelessly transmit the command to the patient's medical device.

5. The system of claim 1, further comprising an antenna is coupled to or within the CP computer, and wherein the CP computer is configured to receive the command from the OHMD via the communication link.

6. The system of claim 5, wherein the antenna is coupled to a port on the CP computer.

7. The system of claim 1, wherein the CP software is further configured to render a graphical user interface (GUI) at a display associated with the CP computer.

8. The system of claim 7, wherein the graphical user interface (GUI) at the display associated with the CP computer is also accessible by the clinician to adjust at least one stimulation parameter of the patient's medical device.

9. The system of claim 7, wherein the GUI at the OHMD enables a subset of functions renderable by the CP software at the GUI at the CP.

10. The system of claim 1, wherein the patient's medical device comprises a plurality of electrodes configured to provide pulses to a tissue of the patient, and wherein the at least one stimulation parameter comprises one or more of a pulse amplitude, a pulse frequency, a pulse duration, an active electrode, and electrode polarity.

11. The system of claim 1, wherein the GUI at the OHMD is further configured to provide status information to the clinician from the patient's medical device.

12. The system of claim 1, wherein the GUI at the OHMD displays the at least one stimulation parameter for the clinician.

13. The system of claim 1, wherein the GUI at the OHMD includes an input interface accessible by the clinician to adjust at least one stimulation parameter.

14. A method of adjusting the stimulation parameters of a patient having a pulse generator medical device, comprising:
coupling an optical head mounted display (OHMD) to a clinician programmer (CP) computer having clinician programming (CP) software;
rendering a graphical user interface (GUI) at the OHMD using the CP software viewable by a clinician wearing the OHMD; and
adjusting at least one stimulation parameter of the patient's medical device using the GUI at the OHMD while the clinician views the patient.

15. The method of claim 14, wherein the OHMD is coupled to the CP via a wireless link.

16. The method of claim 14, wherein adjusting the least one stimulation parameter comprises forming a command and transmitting the command to the patient's medical device.

17. The method of claim 16, wherein the command is transmitted from an antenna in the OHMD.

18. The method of claim 17, wherein the command is transmitted from the OHMD to the CP computer, and wherein the command is further transmitted to the patient's medical device from the antenna, wherein the antenna is coupled to or within the CP computer.

19. The method of claim 14, wherein the CP software is further configured to render a graphical user interface (GUI) at a display associated with the CP computer.

20. The method of claim 14, further comprising rendering a graphical user interface (GUI) at a display associated with the CP computer, and adjusting at least one stimulation parameter of the patient's medical device using the GUI at the display associated with the CP computer.

21. The method of claim 14, wherein the patient's medical device comprises a plurality of electrodes configured to provide pulses to a tissue of the patient, and wherein the at least one stimulation parameter comprises one or more of a pulse amplitude, a pulse frequency, a pulse duration, an active electrode, and electrode polarity.

22. The method of claim 14, wherein adjusting the at least one stimulation parameter of the patient's medical device using the GUI at the OHMD comprising touching a touch surface of the OHMD.

23. The method of claim 14, wherein adjusting the at least one stimulation parameter of the patient's medical device comprising use of a voice or motion input of the GUI of the OHMD.

24. The method of claim 14, wherein the clinician wears the OHMD as eyeglasses.

25. A non-transitory computer-readable media storing instructions that when executed on a computer cause the computer to:
render a graphical user interface (GUI) at an optical head mounted display (OHMD) coupled to the computer, wherein the GUI allows a clinician wearing the OHMD to adjust at least one stimulation parameter of a patient's medical device.
